(19) United States
(12) Patent Application Publication
(10) Pub. No.: US 2008/0090874 A1
(43) Pub. Date: Apr. 17, 2008

(54) Treatment of breakthrough pain in patients suffering from chronic low back pain

(76) Inventor: John C. Messina JR., Downingtown, PA (US)

Correspondence Address: CIMA;LERNER, DAVID ET AL, 600 SOUTH AVENUE WEST, WESTFIELD, NJ, 07090, US

(21) Appl. No.: 11/899,967

(22) Filed: Sep. 6, 2007

Related U.S. Application Data
(60) Provisional application No. 60/845,953, filed on Sep. 20, 2006.

(51) Int. Cl. A61K 31/445; A61P 21/00

(52) U.S. Cl. 514/329

(57) ABSTRACT

Fentanyl containing dosage forms and methods using same are described. These dosage forms include substantially less fentanyl by weight than know oral formulation and have advantages in terms of cost and side effects. These dosage forms are intended for oral administration of fentanyl across the oral mucosa and for the treatment of breakthrough pain in patients who have and are undergoing treatment for chronic lower back pain.

### CROSS-REFERENCE TO RELATED APPLICATIONS

This application claims the benefit of the filing date of U.S. Provisional Patent Application No. 60/845,953 filed Sep. 20, 2006, the disclosure of which is hereby incorporated herein by reference.

### BACKGROUND OF THE INVENTION

Fentanyl (CAS Registry No. 437-38-7) N-phenyl-N-[1-(2-phenyl-ethyl)-4-piperidinyl] propanamide and its salts, in particular its citrate salt (CAS Registry No. 990-73-8) are opiates, controlled substances, and extremely potent narcotic analgesics. Fentanyl and its citrate salt are currently marketed by a number of companies in a number of delivery formats. Fentanyl citrate, for example, is available as an injectable and an oral lozenge on a stick, the latter sold under the trade name ACTIQ.

A review of the package insert information for ACTIQ sold by Cephalon, Inc., 145 Brandy Wine Parkway West, Chester, Pa. 19380, available in the Physician's Desk Reference, 57th ed. 2003 at page 1184, brings instant perspective on the seriousness of the afflictions of the patients who take it. According to its label, ACTIQ “is indicated only for the management of break-through cancer pain in patients with malignancies who are already receiving and who are tolerant to opiate therapy for their underlying persistent cancer pain.” (Id., emphasis in original). The text of the ACTIQ label is hereby incorporated by reference.

U.S. Pat. No. 6,200,604, which issued Mar. 13, 2001 to CIMA LABS INC., 10000 Valley View Road, Eden Prairie, Minn. 55344, exemplifies two fentanyl formulations each containing 36% effervescence and 1.57 milligrams of fentanyl salt. See example I thereof, col. 5, ln. 60 through col. 6, ln. 30. The '604 patent describes the use of, amongst other things, effervescence as a penetration enhancer for influencing oral drug absorption. See also U.S. Pat. Nos. 6,759,059 and 6,680,071. See also Brendenberg, S., 2003 New Concepts in Administration of Drugs in Tablet Form: Formulation and Evaluation of a Sublingual Tablet for Rapid Absorption, and Presentation of an Individualized Dose Administration System, Acta Universitiatis Upsaliensis. Comprehensive Summaries of Uppsala Dissertations from the Faculty of Pharmacy, 287, 83 pp. Uppsala ISBN 91-554-5600-6.

U.S. Patent Application Publication No. 2005/0169989 A1 also describes the use of certain fentanyl formulations, including those generally described herein, to achieve pain reduction using lower doses which nonetheless can provide similar levels of, for example, Cₘₐₓ. Methods of treatment described therein include back pain, lower back pain, joint pain, any form of arthritic pain, pain from trauma or accidents, neuropathic pain, surgical or postoperative pain, pain from a disease or condition other than cancer, cancer pain and in particular breakthrough pain as a result of cancer. However, that application does not discuss the unique problems of treating breakthrough pain in patients with chronic lower back pain.

### SUMMARY OF THE INVENTION

The present invention relates to an orally disintegrable/dissolvable dosage forms including fentanyl, or one or more of its pharmaceutically acceptable salts (where “fentanyl” is recited herein, it should be assumed to include all pharmaceutically acceptable salts unless the context suggests otherwise), methods of making such dosage forms, methods of using such dosage forms to treat breakthrough pain in patients already suffering with chronic lower back pain and uses for the manufacture of a medicament useful for treating breakthrough pain in patients with chronic lower back pain. In one embodiment, the amount of fentanyl administered orally for these treatments are at least about 45% less fentanyl when compared to noneffervescent lollipop formulations (both lozenge and pressed tablets) currently available. Despite the lower dose, these orally disintegrable dosage forms of the invention should have a Cₘₐₓ which is at least comparable to other dosage forms containing much more, e.g., about twice as much drug. “Comparable” in this context means that the Cₘₐₓ of a dosage form of the present invention is at least about 75% that of ACTIQ having about twice as much fentanyl. Thus, if a 400 microgram tablet in accordance with the present invention was compared to a 400 microgram ACTIQ lollipop, and both were compared to an 800 microgram ACTIQ lollipop, the tablet in accordance with the present invention would have a Cₘₐₓ which is at least about 75% to about 125% of the Cₘₐₓ of the 800 microgram ACTIQ formulation. The 400 microgram ACTIQ formulation will have a much lower Cₘₐₓ. This is true for doses of up to about 800 micrograms based on the weight of fentanyl in free form. Note that “about” in this context (doses) means ±10%. Thus, about 100 to about 800 μg is 90 to 880 μg. More preferably, “comparable” in the context of the invention may also mean that the Cₘₐₓ of a dosage form of the present invention is between about 80 and about 120% that of ACTIQ having about twice as much fentanyl by weight.

“Oral dosage form” in the context of the invention preferably includes orally disintegrable/dissolvable tablets, capsules, caplets, lollipop-like lozenges and the like. Preferably, these dosage forms are effervescent tablets. In addition, they may include a pH adjusting substance and a disintegrant.

In another embodiment, the present invention contemplates doses of about 25, 50, 75, 90, 100, 200, 300, 400, 500, 600, 700, 800, 900, 1000, 1200, 1400, 1500, 1600, 1800, and 2000 micrograms of fentanyl, again based on the weight of free base material. Preferred ranges include, without limitation, about 25 to about 90 micrograms, about 25 to about 75 micrograms, about 100 to about 800 micrograms, about 1400 to about 2000 micrograms, about 25 to about 2000 micrograms, and about 50 to about 1800 micrograms. In a particularly preferred embodiment, the specific doses and/or doses falling within these specific ranges are administered by placing the dosage form under the tongue or between the gum and cheek of a patient in need thereof and leaving it there where it completely dissolves/disintegrates. The dosage form in this embodiment is an effervescent dosage form including a pH adjusting substances and a disintegrant and is administered to a person with chronic low back pain (also referred to as chronic lower back pain) to treat episodes of breakthrough pain or any other type of pain. Chronic lower back pain, in this context, is a condition for which a patient is under care from a medical practitioner. This can include receiving regular treatment with medication, including opiates such as fentanyl, to provide a consistent level of pain relief or management. Breakthrough pain is a sporadic and often sudden significant increase pain levels which cannot be managed by the normal regular pain management strategy.

Another embodiment of the present invention comprises treating chronic lower back pain with a dose of between about 25 and about 90 micrograms of fentanyl, more preferably about 25 to about 75 micrograms of fentanyl, to a patient in need thereof, a tablet in accordance with the invention for transmucosal administration, at least once a day. In another embodiment, the patient is dosed with this dose between once and four times a day. Dosage forms containing about 25 to about 90 micrograms of fentanyl (measured as the free base form or a corresponding amount of a salt) are also contemplated, as are dosage forms of 1400, 1500, 1600, 1800, and 2000 micrograms.

In another embodiment, the doses used to treat such breakthrough pain provides a ratio of Cₘₐₓ to dose, over the dosage range of 90 to 880 micrograms, of between about 2.0 and about 4.0 picograms/mL/microgram. That is picograms of fentanyl base per mL of serum or a proportionate amount if determined in blood or other fluid, normalized per microgram of the dose. More preferably, the ratio is about 2.5 to about 3.5 and even more preferably between about 2.7 and about 3.5 picograms/mL/microgram. These ranges are based on mean data calculated for at least 10 patients in an appropriate clinical trial. In contrast, testing has established that ACTIQ provides a ratio of about 1.4 picograms/mL/microgram. Thus for dosage forms containing the same amount of fentanyl, the present invention can provide about twice the Cₘₐₓ, if not more, up to doses of 880 micrograms, e.g., about 800 micrograms using the invention.

Also contemplated as another aspect of the invention are methods of treating patients experiencing lower back pain and in particular treating breakthrough pain in patients suffering form chronic lower back pain. A preferred method includes the steps of administering to a patient in need thereof any orally disintegrable effervescent tablet disclosed herein for buccal, gingival or sublingual administration, which includes a dose of fentanyl of between 25 and about 2000 micrograms and, in particular, about 100-800 micrograms (measured as a free base), and holding the dosage form in the mouth of the patient for a time sufficient to allow transport of said dose (or a therapeutically significant and/or effective portion thereof) from the oral cavity to the blood stream. In another embodiment, the period of time that the dosage form is held in the mouth is between about 5 minutes and about 30 minutes, more preferably 10 minutes to about 30 minutes, most preferably about 10 minutes to about 20 minutes. The dosage form in this embodiment is an effervescent dosage form including a pH adjusting substances and a disintegrant. The method also preferably includes the step of holding the dosage form in the mouth, substantially without moving it within the oral cavity. Preferably, the patient is instructed, trained or watched to ensure that the dose is not swallowed and instead to the extent practicable, the fentanyl enters the body through one or more of the surfaces within the mouth and oral cavity.

In particular, the dosage form used in these methods comprise an effervescent couple in amount of about 5 to about 85% by weight of the dosage form, a pH adjusting substance in an amount of about 0.5 to about 25% by weight of the dosage form and a disintegrant in an amount of about 0.25 to about 20% by weight of the dosage form. The dosage form is suitable for delivery of said fentanyl across the oral mucosa of a patient. By “providing,” “administering,” or like terms, it is understood that removing a dosage form from a package or having someone hand out or dispense such a dosage form may be included. The method, and indeed these terms, certainly include placing the dosage form in the mouth of the patient between the cheek and the upper or lower gum, or under the tongue, for a time sufficient to deliver a therapeutically effective amount of said fentanyl across said oral mucosa and/or for a period of between about 5 and about 30 minutes.

In another embodiment, the method involves selecting a relatively low does to use to treat the patient. If that dose is insufficient, the patient is titrated until an “effective dose” is found for that patient. An “effective dose” in this regard is a dose which provides breakthrough pain relief within 30 minutes of the dosage form being placed in the mouth for about 66% or more of the breakthrough pain episodes experienced by that patient, without realizing unacceptable adverse results.

Indeed, in one embodiment, the present invention is a method of treating breakthrough back pain in a patient with chronic lower back pain who is in need thereof comprising the steps of providing to that person an effective dose of fentanyl as described above, and placing the dosage form in the mouth of a patient for buccal, gingival or sublingual delivery of the fentanyl, and maintaining the dosage form in the mouth for a time which is a period of 5 to 30 minutes.

### BRIEF DESCRIPTION OF THE DRAWINGS

FIG. 1 illustrates the effect of titrated doses of fentanyl buccal tablets (FBT) in accordance with the invention against placebo on pain intensity difference (PID) scores in patients with breakthrough pain and chronic low back pain. The solid squares represent data for FBTs and shaded circles represent data for placebo.

FIG. 2 illustrates the proportion of breakthrough pain episodes with ≧33% decrease in pain intensity (PI) scores following titrated administration of FBT or placebo. The solid bars represent data for FBT and the shaded bars represent data for placebo.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a tablet or other dosage form of the invention as, for example, containing particles having a certain particle size or distribution or a certain type of, for example, a nondirect compression sugar, it may be difficult to tell from the final dosage form that the recitation is satisfied.

However, such a recitation may be satisfied if the materials used prior to final blending and tablet formulation, for example, meet that recitation. In another example, while it might be difficult to know the weight gain of a coated API-containing particle or its particle size distribution as it actually exists in a finished dosage form, if it is determined that the coated API-containing particles used to make the dosage form, prior to, in the case of a tablet, for example, a final blending and compression step, did exhibit the desired coating level and/or particle size, that is sufficient. Indeed, as to any property of a dosage form which cannot be ascertained from the dosage form directly, it is sufficient if that property resides in the formulation just prior to producing a dosage form therefrom.

The present invention includes, in one aspect, a method of administering fentanyl to a patient experiencing breakthrough pain in patients under treatment for chronic lower back pain is another aspect of the invention. This method can comprise the steps of contacting the oral mucosa of a patient in need thereof with an orally disintegrable, dosage form including an effervescent couple, a pH adjusting substance and a disintegrant. The dosage form includes a dose of fentanyl or a salt thereof. In preferred embodiments, the dosage form is capable of providing: a Tₘₐₓ of 1.5 hours or less; and/or a ratio of Cₘₐₓ to dose of between about 2.0 and about 4.0, more preferably between about 2.3 and about 3.5 and most preferably between about 2.7 and about 3.5 picograms/mL/microgram; and/or which includes at least 45% less fentanyl than would otherwise be prescribed using commercially known delivery formats for fentanyl. The dosage form is held in contact with the oral mucosa of the patient for a time sufficient to allow transport of a therapeutically significant or effective portion of the fentanyl, preferably more than 75%, more preferably more than 80% and most preferably 90% or more of the dose, from the oral cavity to the blood stream across the oral mucosa, or a period of between about 5 minutes and about 30 minutes, more preferably 10 minutes to about 30 minutes, most preferably about 10 minutes to about 20 minutes.

Another aspect of the invention provides a dosage form for use in a method of administering fentanyl to a patient experiencing breakthrough pain in patients under treatment for chronic lower back pain which comprises between about 25 and about 2000 micrograms, more preferably about 100 and about 800 micrograms of fentanyl per dosage form, calculated as fentanyl free base. A fentanyl salt, when used, is used in an amount providing an equivalent amount of fentanyl free base by weight. The dosage form is suitable for buccal, sublingual or gingival administration. The dosage form, when properly administered by contacting it to the oral mucosa for a sufficient time, is capable of providing a Cₘₐₓ which is at least about 75 to about 1256, more preferably between about 80 and about 1206, and most preferably between about 85% to about 115% that of an ACTIQ® formulation wherein the latter includes at least 806 more fentanyl by weight. Preferably, this dosage form also includes at least one pH adjusting substance and at least one effervescent couple in an amount which is sufficient to provide the stated Cₘₐₓ. Even more preferably, the dosage form further comprises at least one excipient in an amount which, in combination with the at least one pH adjusting substance and/or at least one effervescent couple is sufficient to provide the desired Cₘₐₓ. Methods of administering these fentanyl dosage forms to a patient experiencing breakthrough pain in patients under treatment for chronic lower back pain is another aspect of the invention.

The use of effervescence and a pH adjusting substance, particularly when combined with a starch glycolate, can provide significant advantages particular in terms of the amount of fentanyl that is required for dosing. It has also been found that certain excipients in combination with effervescent couples and pH adjusting substances can provide even better, and very unexpected, results.

Determining whether or not a particular formulation is capable of achieving the results described herein, one need only undertake a routine human clinical study of that formulation in at least 10 patients. The appropriate clinical study would use any of the traditional models. Examples of appropriate studies follow:

Clinical Study Design and Conduct

Breakthrough pain (BTP) is a transitory, moderate-to-severe flare of pain in patients with otherwise stable, controlled persistent pain. The use of as-needed, shortacting opioids to treat BTP during long-term opioid therapy for low back pain is now widely accepted in medically ill patients with chronic pain.

Eligible patients entered an open-label phase and were individually titrated to an effective fentanyl buccal tablets (FBT) dose (100, 200, 400, 600, or 800 μg tablets providing BTP relief by 30 min for ≧⅔ episodes without unacceptable adverse events; AEs). Tablets were of the formulations described in Examples 7, 6, 9, 10, and 11 respectively. Patients finding an effective FBT dose entered the double-blind phase and were randomly assigned to 1 of 3 sequences in which 9 BTP episodes were treated with 6 FBT and 3 placebo doses in different orders. Self-reported pain intensity was measured on an 11-point scale (0=no pain; 10=worst pain) at 5, 10, 15, 30, 45, 60, 90, and 120 minutes and the pain intensity differences (PIDs) between each time point and pre-treatment pain were calculated. The primary endpoint was the sum of PIDs for the first 60 minutes (SPID₆₀); secondary endpoints included PIDs at each time point, the proportion of treated BTP episodes with a ≧33% reduction in PI score, patient assessment of time to meaningful PR, and use of usual BTP medication.

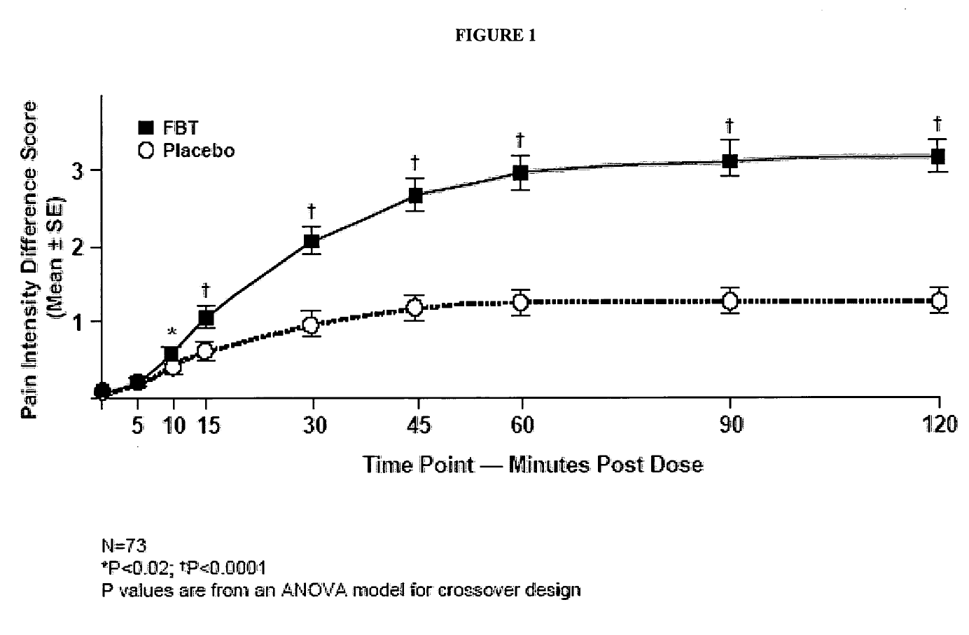

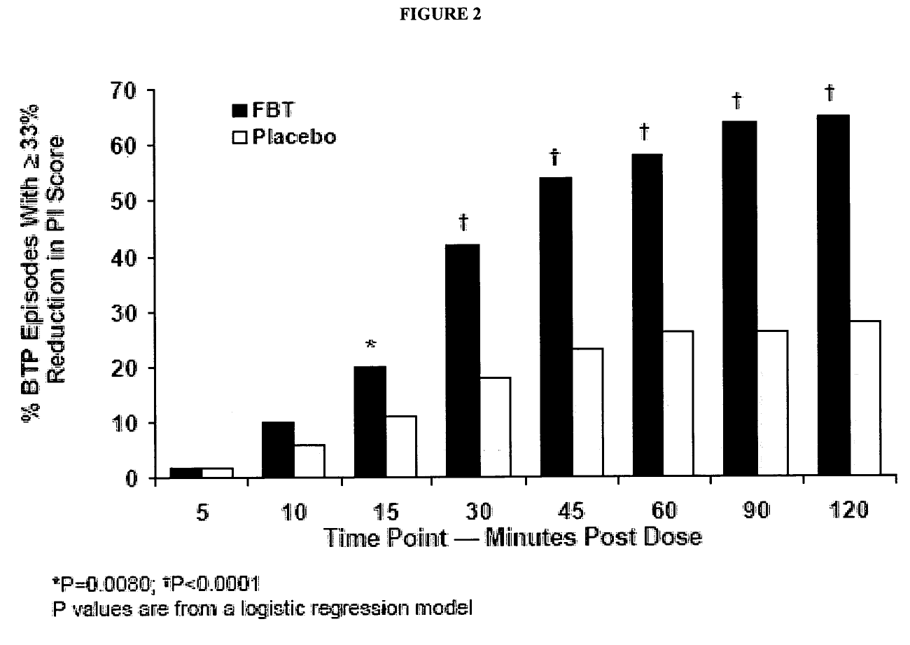

Of 105 patients enrolled, 77 completed the open-label phase and 75 completed double-blind phase; 73 patients were evaluated for efficacy. The primary endpoint (SPID₆₀) differed significantly between FBT and placebo (P<0.0001); significant differences in PIDs started at 10 minutes (P<0.02) and occurred at all subsequent time points (P<0.0001; FIG. 1). The % of BTP episodes with a ≧33% decrease in PI score was greater for FBT vs placebo, beginning at 15 minutes (20% vs 11%, P<0.01; FIG. 2) and continuing for all subsequent time points (P<0.0001). Patients experienced meaningful PR for 70% (289/413) of BTP episodes treated with FBT vs 30% (63/207) for placebo (P<0.0001). Meaningful PR was achieved by 30 minutes in 38% of BTP episodes treated with FBT (155/413) vs 17% (34/207) for placebo. Patients were approximately 4 times as likely to require supplemental opioids for a BTP episode following placebo than FBT (risk ratio 0.22 [95% CI: 0.13, 0.35]). AEs were typical for opioids and were more frequent during the titration phase (57%) than double-blind phase (34%); 11 of 12 patients who withdrew due to AEs did so during the titration phase. Five patients reported ≧1 mild AE related to the FBT application site. Thus, FBT is safe and efficacious for BTP in opioid-treated patients with chronic back pain. Onset of effect is rapid compared to the usual time course of oral medications.

Dosage Forms and Methods

Any formulation which contains sufficient effervescent material and pH adjusting substance, preferably with a suitable disintegrant, which is capable of providing a dosage form useful in buccal, gingival, or sublingual administration of fentanyl at dose levels which are contemplated herein, and useful in treating breakthrough pain in patients undergoing treatment for chronic lower back pain, are contemplated. Formulations in the '604 patent which included lactose monohydrate in an amount of greater than 20% and/or microcrystalline cellulose in an amount of at least about 20% and cross-linked PVP in an amount of 5% or more are believed to be unable to provide formulations having the desirable linear behavior of dose and Cₘₐₓ of the levels discussed herein, despite the presence of a pH adjusting substance and an effervescent couple. The formulations in the '604 patent also have more than 880 μg of fentanyl.

A preferred effervescent, orally disintegrable dosage form in accordance with the present invention is one that includes, based on the weight of the free base material, about 25, 50, 100, 200, 300, 400, 500, 600, 800, 1000, 1200, 1400, 1600, 1800 and 2000 micrograms of fentanyl. Most preferably, for dosage forms contain about 100-800 micrograms of fentanyl (calculated as free base), or a proportionate weight of one of its pharmaceutically acceptable salts. In addition, these numbers are meant to include normal processing variabilities such as content uniformity, etc. Particularly preferred doses are about 25 micrograms, about 50 micrograms, about 100 micrograms, about 200 micrograms, about 300 micrograms, about 400 micrograms, about 600 micrograms and about 800 micrograms, respectively.

It is preferred that the mean particle size as determined by a laser diffraction technique of fentanyl used in the present formulation range from between about 0.2 to about 150 microns, more preferably from between about 0.5 to about 100 and most preferably from between about 1 to about 20 microns.

As an effervescent agent or effervescent couple, any known combination may be used. These include those described in U.S. Pat. No. 5,178,878 and U.S. Pat. No. 5,503,846, the texts of which are hereby incorporated by reference to the extent they discuss various effervescent couples and constructions of same. Effervescent couples generally are water- or saliva-activated materials usually kept in an anhydrous state with little or no absorbed moisture or in a stable hydrated form. Typically these involve at least one acid source and at least one source of a reactive base, usually a carbonate or bicarbonate. Each of the components of the effervescent couple may be any which are safe for human consumption.

The acids generally include food acids, acid anhydrides and acid salts. Food acids include citric acid, tartaric acid, malic acid, fumeric acid, adipic acid, ascorbic acid and succinic acid. Acid anhydrides or salts of these acids may be used. Salts in this context may include any known salt but in particular, sodium, dihydrogen phosphate, disodium dihydrogen phosphate, acid citrate salts and sodium acid sulfate. Bases useful in accordance with the invention typically include sodium bicarbonate, potassium bicarbonate and the like. Sodium carbonate, potassium carbonate, magnesium carbonate and the like may also be used to the extent they are used as part of an effervescent couple. However, they are more preferably used as a pH adjusting substance. Preferably, stoichiometric equivalent amounts of acid, acid anhydride or acid salt and base are used. It is possible, however, that some excess of acid or base be used. However, care should be exercised when so formulating a formulation, particularly in view of the overall pH adjusting effect of such components, if any. An excess could affect absorption.

The amount of effervescent material useful in accordance with the present invention is an effective amount and is determined based on properties other than those which would be necessary to achieve disintegration of the tablet in the mouth. Instead, effervescence is used as a basis for enhancing transmission of the fentanyl across mucosal membranes via buccal, gingival or sublingual administration in the oral cavity. Accordingly, the amount of effervescent couple should range from between about 5 to about 85 percent, more preferably between about 15 to 60 percent, even more preferably between about 30 and 45 percent and most preferably between about 35 to about 40 percent, based on the weight of the total formulation. Of course, the relative proportion of acid base will depend upon the specific ingredients (for example, whether the acid monoprotic, diprotic or triprotic) relative molecular weights, etc. However, preferably, a stoichiometric amount of each is provided although, of course, excesses are acceptable.

Preferably, formulations in accordance with the present invention include at least one pH adjusting substance. Without wishing to be bound by any particular theory, this permits a drug which is susceptible to changes in ionization state can be administered by ensuring the proper conditions for its dissolution as well as transmission across one or more of the membranes or tissues within the oral cavity such as across the oral mucosa. If the ideal conditions for transmission of a particular drug are basic, the addition of a sufficient excess of suitably strong acid as part of the manufacture of an effervescent couple or as a pH adjusting substance may not be indicated. The selection of another pH adjusting substance such as, for example, anhydrous sodium carbonate which operates separate and apart from the effervescent agents would be preferred.

pH adjusting substances in accordance with the present invention can be used to provide further permeation enhancement. The selection of the appropriate pH adjusting substance will depend on the drug to be administered and, in particular, to the pH at which it is ionized or unionized, and whether the ionized or unionized form facilitates transmission across the oral mucosa. With regard to fentanyl and its salts, a basic substance is preferred for the delivery of fentanyl. pH adjusting substances in accordance with the present invention can include, without limitation, any substance capable of adjusting the localized pH to promote transport across the membranes in the oral cavity in amounts which will result in a pH's generally ranging from between about 3 to 10 and more preferably between about 4 to about 9. The pH is the “localized pH” at the microenvironment in the mouth of a patient at the surface contact area of the oral mucosa and the dosage form or any portion thereof (such as when it disintegrates). For purposes of this invention, the localized pH can be determined as follows: to characterize the dynamic pH changes displayed by the tablets in question, an in vitro pH measurement was used. The method consists of using 0.5-10 mL of phosphate buffered saline in an appropriately sized test tube or similar vessel. The amount of media is dependent on the tablet size and dosage. For example, when measuring the pH profile for fentanyl tablets, a volume of 1 mL was used for tablets which weighed 100 mg. Immediately upon tablet contact with the media, the pH profile of the solution is monitored as a function of time, using a micro-combination pH electrode. Preferably, the materials which can be used as pH adjusting substances in accordance with the present invention include carbonates such as sodium, potassium or calcium carbonate or a phosphate such as calcium or sodium phosphate. Most preferred is sodium carbonate. The amount of pH adjusting substance useful in accordance with the present invention can vary with the type of pH adjusting substance used, the amount of any excess acid or base from the effervescent couple, the nature of the remaining ingredients and, of course, the drug which, in this case, is fentanyl.

Most preferably the amount of pH adjusting substance will range from between about 0.5 to about 25 percent, more preferably between about 2 to about 20 percent, even more preferably between about 5 to about 15 percent and most preferably between about 7 to about 12 percent by weight based on the weight of the total formulation. The most preferred pH adjusting substance is a carbonate, bicarbonate, or phosphate. Also preferred are those pH adjusting substances which, when provided in a suitable amount, can provide a change in the localized pH of at least about 0.5 pH units, more preferably about 1.0 pH units and even more preferably about 2.0 pH units when compared to an otherwise identical formulation without the pH adjusting substance.

Any filler or any amount of a filler may be used as long as the resulting dosage forms achieve the results described herein. Most preferred amongst the fillers are sugar and sugar alcohols and these may include non-direct compression and direct compression fillers. Non-direct compression fillers generally, at least when formulated, have flow and/or compression characteristics which make them impractical for use in high speed tableting process without augmentation or adjustment. For example, a formulation may not flow sufficiently well and therefore, a glidant such as, for example, silicon dioxide may need to be added.

Direct compression fillers, by contrast, do not require similar allowances. They generally have compressibility and flowability characteristics which allow them to be used directly. It is noted that, depending upon the method by which formulations are made, non-direct compression fillers may be imparted with the properties of direct compression fillers. The reverse is also true. As a general matter, non-direct compression fillers tend to have a relatively smaller particle size when compared to direct compression fillers. However, certain fillers such as spray dried mannitol have relatively smaller particle sizes and yet are often directly compressible, depending upon how they are further processed. There are also relatively large nondirect compression fillers as well.

Fillers that are preferred in accordance with the present invention include mannitol, lactose, sorbitol, dextrose, sucrose, xylitol and glucose, to the extent their use can provide the results described herein. More preferably in accordance with the present invention, the filler is not lactose monohydrate used in an amount of 20% or more based on the weight of the formulation and even more preferably no lactose monohydrate is used. Most preferred in accordance with the present invention, spray dried mannitol is used. The amount of filler can range from 10 to about 80% and more preferably about 25 to about 80%, most preferably 35 to about 60% by weight of the formulation.

Disintegrants may also be used in accordance with the present invention so long as they permit or even facilitate the dose reductions, linearity and/or ratio of Cₘₐₓ and dose as described herein. These may also include binders that have disintegrating properties. Disintegrants in accordance with the present invention can include microcrystalline cellulose, cross-linked polyvinyl pyrrolidone (PVP-XL), sodium starch glycolate, croscaramellose sodium, cross-linked hydroxypropyl cellulose and the like. Of course, the selection of the disintegrant depends upon whether or not, in a given system, the results described herein may be obtained. More preferably, the formulation will be free of more than about 20% microcrystalline cellulose and cross-linked polyvinyl pyrrolidone in an amount of about 5% or more, especially in a formulation that includes in additional 20% lactose monohydrate. Most preferred for use as a disintegrant is a starch glycolate and in particular sodium starch glycolate. Indeed, it has been found that the use of sodium starch glycolate in the formulations of the present invention can provide significant improvement in the degree of dose reduction, while still providing a comparable Cₘₐₓ, when compared to effervescent formulations which include pH adjusting substances and other disintegrants. A particularly useful sodium starch glycolate is GLYCOLYS® (standard grade) available from Roquette of Lestrem France. Indeed, it is even more preferred that the formulation include neither microcrystalline cellulose nor cross-linked PVP.

The amount of disintegrant will vary with known factors such as, the size of the dosage form, the nature and amounts of the other ingredients used, etc. However, in general the amount should range from between about 0.25 to about 20% by weight of the final formulation, more preferably between about 0.5 to about 15% w/w, even more preferably 0.5 to about 10% w/w and even more preferably between about one and about eight percent by weight. This is again based on the weight of the finished formulation.

Also generally useful in accordance with the present invention is a tableting or ejection lubricant. The most common known lubricant is magnesium stearate and the use of magnesium stearate is preferred. Generally, the conventional wisdom behind tableting lubricants is that less is more. It is preferred in most circumstances that less than about one percent of a tableting lubricant be used. Typically, the amount should be half a percent or less. However, the amount of magnesium stearate used can be greater than 1.0%. Indeed, it is preferably greater than about 1.5% and most preferably between about 1.5% and about 3%. Most preferred is the use of about 2% magnesium stearate. Other conventional tableting lubricants such as, for example, stearic acid, calcium stearate and the like may also be used in place of some or all of the magnesium stearate.

Effervescent tablets in accordance with the present invention can be relatively soft or robust. They can, for example, be manufactured in accordance with the methods described in U.S. Pat. No. 5,178,878 and will have a hardness of generally less than about 15 Newtons. Unlike the formulations described in the '878 patent, the active ingredient here will not necessarily be coated with a protective material. Indeed, preferentially, the fentanyl active will not be coated. When tablets as soft and pliable/friable as these are produced, they may be advantageously packaged in a blister package such as found in U.S. Pat. No. 6,155,423. They may also be robust with a hardness of greater than about 15 Newtons, manufactured in accordance with the procedures set forth in U.S. Pat. No. 6,024,981. In a preferred embodiment, the fentanyl dosage forms of the invention are provided in a blister package which is child resistant. See for example U.S. Pat. No. 6,155,423 to Katzner et al., issued Dec. 5, 2000 and assigned to CIMA LABS INC., the text of which is hereby incorporated by reference. Most preferably, the package meets the standards set forth in 16 U.S.C. § 1700.15 and 0.20 (2003). Packages also preferred include those commonly referred to in the industry as so-called “F1” and “F2” packages. “F1” packages are most preferred.

Tablets in accordance with the present invention may be designed slightly differently for buccal, gingival, or sublingual administration. In each instance, however, the in mouth disintegration time/dissolution (dwell time) achieved by the formulations is preferably less than about 30 minutes and most preferably, about 20 minutes or less. It is usually more than five minutes, most often 10 minutes or more. This is a subjective determination based on the response of the patient.

In a particularly preferred aspect of this embodiment of the present invention, the formulations described above do not include an amount of lactose monohydrate and/or cross-linked PVP which render it incapable of obtaining a dose reduction relative to ACTIQ® of at least about 45% fentanyl by weight. In particular, it is preferred that no more than about 10% by weight of the formulation be lactose monohydrate or microcrystalline cellulose and no more than about 4% crosslinked PVP. More preferably, the formulation is free from all but incidental amounts of these excipients. Most preferred in accordance with the present invention are the use of sodium starch glycolate as a disintegrant and mannitol as a filler. Most preferred filler includes spray dried mannitol.

The formulations in accordance with the present invention can include other conventional excipients in generally known amounts to the extent they do not detract from the advantages described herein. These can include without limitation binders, sweeteners, coloring components, flavors, glidants, lubricants, preservatives, disintegrants, and the like.

Tablets, a preferred dosage form in accordance with the present invention, can be made by any known tableting technique. However, preferably, the materials used are dry blended and directly compressed. While the tablets may result from granulation, this is not preferred. Of course, particular excipients and materials used in formulations in accordance with the present invention may be wet or dry granulated. For example, granulated mannitol could be used as a filler. It may also be desirable to granulate or pre-mix some portion of the formulation prior to final blending and compression. The materials in question are preselected to provide the right dose and content uniformity and the dose reduction, Cₘₐₓ/dose ratio and/or dose linearity described herein. Thus, an appropriate amount of an effervescent couple, a suitable and appropriate pH adjusting substance and an appropriate disintegrant are selected, provided in predetermined amounts and formulated to dosage forms, preferably tablets.

The preferred pH adjusting substances are carbonates, bicarbonates, or phosphates, the preferred disintegrant is a starch glycolate. The amounts used of each are described elsewhere herein. However, preferably, the disintegrant is selected and provided in an amount which can provide a further dose reduction in the amount of fentanyl used when compared to an otherwise identical formulation containing an effervescent couple and a pH adjusting substance without the disintegrant. The pH adjusting substance preferably is selected and provided in an amount sufficient which is capable of providing a change in localized pH of at least 0.5 pH units, more preferably 1.0 pH unit and most preferably about 2.0 pH units or more. While tablets may be compressed to any hardness and/or friability, same must be accomplished without adversely affecting dwell times and drug release and transmission across the oral mucosa. Where possible, it is desirable to provide fentanyl dosage forms as compressed tablets having a hardness of between about 5 and about 100 Newtons, more preferably between about 10 and about 50 Newtons.

The dosage forms in accordance with the present invention may be used to treat any type of back pain but in particular lower back pain and even more particularly, breakthrough pain which is experienced in patients suffering from and even more preferably being treated for, chronic lower back pain. As with all opiates, fentanyl products and particularly those of the present invention should always be taken in consultation with a doctor and under a physician's strict care and supervision. The general directions for the use of the ACTIQ product as found in the previously mentioned label found in the Physician's Desk Reference and the warnings and contraindications therein are broadly applicable to the use of dosage forms in accordance with the present invention. This includes generally titrating patients with lower doses before dose escalation.

In particular, one method in accordance with the present invention for treating episodes of breakthrough back pain comprises the steps of providing an initial dose of fentanyl which is typically about 25 to about 100 micrograms calculated as the fentanyl free base or an equivalent amount of the salt. This is often done using a dosage form which comprises an effervescent couple in an amount of about 5 to about 85% by weight of the dosage form, a pH adjusting substance in an amount of about 0.5 to about 25% by weight of the dosage form and a disintegrant in an amount of between about 0.25 and about 20% by weight of the dosage form. The dosage form is suitable for delivery of the fentanyl across the oral mucosa of the patient. More specifically, the dosage form is removed from the packaging either by the patient or by someone else and thereafter it is placed in the patient's mouth for a time sufficient to deliver a therapeutically effective amount across the oral mucosa or a period of between about 5 and about 30 minutes. The packaging can be and preferably is a childproof or child resistant packaging; a so-called F1 package.

Thereafter, the patient would be titrated by increasing the dose as necessary to find an effective dose which balances relief from breakthrough pain with potentially unacceptable adverse results. In particular, one method of the present invention involves treating a patient suffering from chronic lower back pain and experiencing breakthrough pain with an effective dose of fentanyl or its salt. In this regard, an effective dose is a dose which provides breakthrough pain relief within 30 minutes of the dosage form being placed in the mouth of the patient about 66% or more of the breakthrough pain episodes experienced by the patient without realizing adverse side effects. Necessarily, the effective dose can be based on past experience with the patient with other drugs and/or other dosage forms containing fentanyl or its salts or based on the results of repeated dosing with a dosage form in accordance with the present invention which, upon monitoring, shows that about two-thirds of the time or more, that dose is satisfactory for providing relief within 30 minutes from breakthrough pain episodes.

In yet another embodiment, the present invention involves a method of treating breakthrough back pain in a person in need thereof suffering from chronic lower back pain comprising the steps of providing to that person an effective dose of fentanyl as described above by placing the dosage form in the mouth of the patient for buccal, gingival or sublingual delivery and maintaining the dosage form in the mouth for a time which is sufficient to allow transfer of the fentanyl to the blood stream across the oral mucosa in a therapeutically significant and/or effective amount as described herein.

The dosage forms in accordance with the present invention are administered by being placed in the mouth of a patient, preferably under the tongue or in between the cheek and gum, where they remain, preferably without being moved, until their dissolution/disintegration is substantially complete and they cease to be recognizable as a dosage form. Preferably, swallowing is minimized to assist in facilitating the maximum transfer of the fentanyl across the adjacent oral mucosa.

In accordance with one preferred aspect of the present invention, the method comprises the steps of treating breakthrough back pain in a person undergoing treatment for chronic lower back pain who is in need thereof comprising the steps of providing to that person an effective dose of fentanyl, in a dosage form as described herein, by placing the dosage form in the mouth of a patient for buccal, gingival or sublingual delivery so as to achieve one or more of the following: 1) the percent of breakthrough pain episodes in which a ≧33% decrease in pain index score is achieved at 15 minutes from dosing; 2) the SPID₆₀ differed statistically significantly between placebo at 10 minutes from dosing; 3) meaningful pain reduction was achieved within 30 minutes in >30% of the breakthrough pain episodes; or 4) patients experienced meaningful pain relief in at least about 60%, more preferably about 70% of breakthrough pain episodes overall.

In a preferred embodiment, a blister package containing a dosage from and in accordance with the present invention should be opened immediately prior to the product's use. The patient should place the dosage form in his or her mouth, preferably between the cheek and the upper or lower gum. The dosage form should not be sucked or chewed. Fentanyl, as with many opiates, is preferably titrated with the initial dose being a relatively low dose. The initial dose for dosage forms for fentanyl formulations in accordance with the present invention, especially those used to treat episodes of breakthrough cancer pain, should be 100 micrograms. The patient should be provided with a limited initial titration supply of 100 microgram dosage forms, thus limiting the number of units in the home during titration. Thereafter, doses may be escalated under a doctor's care.

This document includes the following documents which are attached and incorporated by reference: U.S. Patent Application Publication No. 2005/0169989 A1; a 12 pg document entitled Fentanyl buccal tablet (FBT) for relief of breakthrough pain in opioid-treated patients with chronic low back pain; and 2 pg document with the same title dated August 4.

### EXAMPLES

Method of Manufacture

In each case for examples 1-7 and 9-11, materials were screened prior to use, charged into a V-blender, or can be blended in any other appropriate low shear blender, and blended for an appropriate time. After discharge from the blender, the materials were compressed on a standard rotary tablet press to a target hardness of 13 Newtons and a target weight of 100 or 200 mg as described in each example.

### Example 1

### Form A

OraVescent ® Fentanyl, 1080 mcg, 5/16″ Tablet, Red

| | QUANTITY |
|---|---|
| COMPONENT NAME | (mg/tab) |
| Fentanyl Citrate, USP | 1.69 |
| Mannitol, USP* | 95.31 |
| Sodium Bicarbonate, USP/EP/JP | 42.00 |
| Citric Acid, USP/EP/JP | 30.00 |
| Sodium Carbonate, USP/NF | 20.00 |
| Sodium Starch Glycolate, NF/EP | 6.00 |
| Magnesium Stearate, NF/EP/JP | 4.00 |
| Red Ferric Oxide, NF | 1.00 |
| TOTAL | 200.00 |

*spray dried (Mannogem EX by SPI Pharma)

### Example 2

### Form C

OraVescent ® Fentanyl, 1300 mcg, 5/16″ Tablet, Red

| | QUANTITY |
|---|---|
| COMPONENT NAME | (mg/tab) |
| Fentanyl Citrate, USP | 2.04 |
| Mannitol, USP* | 94.96 |
| Sodium Bicarbonate, USP/EP/JP | 42.00 |
| Citric Acid, USP/EP/JP | 30.00 |
| Sodium Carbonate, USP/NF | 20.00 |
| Sodium Starch Glycolate, NF/EP | 6.00 |
| Magnesium Stearate, NF/EP/JP | 4.00 |
| Red Ferric Oxide, NF | 1.00 |
| TOTAL | 200.00 |

*spray dried

### Example 3

### Form D

OraVescent ® Fentanyl, 810 mcg, 5/16″ Tablet, Yellow

| | QUANTITY |
|---|---|
| COMPONENT NAME | (mg/tab) |
| Fentanyl Citrate, USP | 1.27 |
| Mannitol, USP* | 95.73 |
| Sodium Bicarbonate, USP/EP/JP | 42.00 |
| Citric Acid, USP/EP/JP | 30.00 |
| Sodium Carbonate, USP/NF | 20.00 |
| Sodium Starch Glycolate, NF/EP | 6.00 |
| Magnesium Stearate, NF/EP/JP | 4.00 |
| Yellow Ferric Oxide, NF | 1.00 |
| TOTAL | 200.00 |

*spray dried

### Example 4

### Form E

OraVescent ® Fentanyl, 270 mcg, 5/16″ Tablet, White

| | QUANTITY |
|---|---|
| COMPONENT NAME | (mg/tab) |
| Fentanyl Citrate, USP | 0.42 |
| Mannitol, USP* | 97.58 |
| Sodium Bicarbonate, USP/EP/JP | 42.00 |
| Citric Acid, USP/EP/JP | 30.00 |
| Sodium Carbonate, USP/NF | 20.00 |
| Sodium Starch Glycolate, NF/EP | 6.00 |
| Magnesium Stearate, NF/EP/JP | 4.00 |
| TOTAL | 200.00 |

*spray dried

### Example 5

OraVescent ® Fentanyl, 500 mcg, 5/16″ Tablet, Orange

| | QUANTITY |
|---|---|
| COMPONENT NAME | (mg/tab) |
| Fentanyl Citrate, USP | 0.79 |
| Mannitol, USP* | 96.21 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| Yellow Ferric Oxide, NF | 0.60 |
| Red Ferric Oxide, NF | 0.40 |
| TOTAL | 200.00 |

*spray dried

### Example 6

OraVescent ® Fentanyl, 200 mcg, 5/16″ Tablet, White

| | QUANTITY |
|---|---|
| COMPONENT NAME | (mg/tab) |
| Fentanyl Citrate, USP | 0.32 |
| Mannitol, USP* | 97.69 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| TOTAL | 200.00 |

*spray dried

### Example 7

OraVescent ® Fentanyl, 100 mcg, ¼″ Tablet, White

| | QUANTITY |
|---|---|
| COMPONENT NAME | (mg/tab) |
| Fentanyl Citrate, USP | 0.16 |
| Mannitol, USP* | 48.84 |
| Sodium Bicarbonate, USP/EP/JP | 21.00 |
| Citric Acid, USP/EP/JP | 15.00 |
| Sodium Carbonate, NF | 10.00 |
| Sodium Starch Glycolate, NF/EP | 3.00 |
| Magnesium Stearate, NF/EP/JP | 2.00 |
| TOTAL | 100.00 |

*spray dried

### Example 8

The materials may be screened prior to use, charged into a V-blender or other appropriate low shear blender, and blended for an appropriate time. After discharge from the blender, the materials may be compressed on a standard rotary tablet press to a target hardness of 13 Newtons and a target weight of 200 mg/tablet.

OraVescent ® Fentanyl, 300 mcg, 5/16″ Tablet, Light Yellow

| | QUANTITY |
|---|---|
| COMPONENT NAME | (mg/tab) |
| Fentanyl Citrate, USP | 0.47 |
| Mannitol, USP* | 97.33 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| Yellow Ferric Oxide, NF | 0.20 |
| TOTAL | 200.00 |

*spray dried

### Example 9

OraVescent ® Fentanyl, 400 mcg, 5/16″ Tablet, Pink

| | QUANTITY |
|---|---|
| COMPONENT NAME | (mg/tab) |
| Fentanyl Citrate, USP | 0.63 |
| Mannitol, USP* | 97.17 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| Red Ferric Oxide, NF | 0.20 |
| TOTAL | 200.00 |

*spray dried

### Example 10

OraVescent ® Fentanyl, 600 mcg, 5/16″ Tablet, Orange

| | QUANTITY |
|---|---|
| COMPONENT NAME | (mg/tab) |
| Fentanyl Citrate, USP | 0.94 |
| Mannitol, USP* | 96.06 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| Yellow Ferric Oxide, NF | 0.60 |
| Red Ferric Oxide, NF | 0.40 |
| TOTAL | 200.000 |

*spray dried

### Example 11

OraVescent ® Fentanyl, 800 mcg, 5/16″ Tablet, Yellow

| | QUANTITY |
|---|---|
| COMPONENT NAME | (mg/tab) |
| Fentanyl Citrate, USP | 1.26 |
| Mannitol, USP* | 95.74 |
| Sodium Bicarbonate, USP/EP/JP | 42.00 |
| Citric Acid, USP/EP/JP | 30.00 |
| Sodium Carbonate, NF | 20.00 |
| Sodium Starch Glycolate, NF/EP | 6.00 |
| Magnesium Stearate, NF/EP/JP | 4.00 |
| Yellow Ferric Oxide, NF | 1.00 |
| TOTAL | 200.00 |

*spray dried

### Example 12

The following materials are weighed and screened.

| | | Qty./Tablet | Qty./Batch |
|---|---|---|---|
| # | Description | (% w/w) | (kg) |
| 1 | Fentanyl Citrate | 0.6285 | 502.8 g* |
| 2a. | Mannitol EZ | 23.875 | 19.1 |
| 2b. | Mannitol EZ | 24.014 | 19.2 |
| 3. | Sodium Bicarbonate, No. 1 | 21.000 | 16.8 |
| 4. | Citric Acid, Anhydrous, | 15.000 | 12.0 |
| | Fine Granular | | |
| 5. | Sodium Carbonate, | 10.000 | 8.000 |
| | Anhydrous | | |
| 6. | Sodium Starch Glycolate | 3.000 | 2.400 |
| 7. | Yellow 10 Iron Oxide | 0.500 | 0.400 |
| 8. | Magnesium Stearate, Non- | 2.000 | 1.600 |
| | Bovine | | |
| | Total | 100.000 | 80.0 |

Transfer Mannitol EZ (2a.) and Yellow 10 Iron Oxide to V-blender and blend for 30 minutes. Discharge and mill preblend. Add the total quantity of preblend, fentanyl citrate, sodium bicarbonate, citric acid, sodium carbonate and sodium starch glycolate to V-blender and blend for 30 minutes. Charge Mannitol (2b) into V-blender and blend for 13 minutes. Charge magnesium stearate into V-blender and blend for 5 minutes. Compress tablets from this final blend. These tablets are ¼″ round, flat faced, white with a beveled edge. They are compressed to an average hardness of 13 Newtons on a 36 station Fette tablet press fully tooled.

We claim:

1. A method of treating breakthrough pain in a patient having chronic lower back pain who is in need of treatment for said breakthrough pain comprising the steps of: placing a tablet comprising about 25 to about 800 micrograms of fentanyl, calculated as fentanyl free base or an equivalent amount of a salt thereof, an effervescent couple in an amount of about 5 to about 85% by weight of the tablet, a pH adjusting substance said adjusting substance selected and provided in an amount capable of providing a change in localized pH of at least 0.5 pH units, and a starch glycolate in an amount of about 0.25 to about 20% by weight of the tablet, into the mouth of a patient in contact with said patient's oral mucosa, and maintaining said tablet in intimate contact with said oral mucosa for a time of between about 5 and about 30 minutes.

2. A dosage form comprising: a tablet comprising about 25 to about 75 micrograms of fentanyl, calculated as fentanyl free base or an equivalent amount of a salt thereof, an effervescent couple in an amount of about 5 to about 85% by weight of the tablet, a pH adjusting substance said adjusting substance selected and provided in an amount capable of providing a change in localized pH of at least 0.5 pH units, and a starch glycolate in an amount of about 0.25 to about 20% by weight of the tablet.
